(19) United States
(12) Patent Application Publication
(10) Pub. No.: US 2025/0189482 A1
(43) Pub. Date: Jun. 12, 2025

(54) METHOD FOR DETERMINING HEROIN

(71) Applicant: UNIWERSYTET LODZKI, Lodz (PL)

(72) Inventors: Lukasz Poltorak, Lodz (PL); Paulina Borgul, Lodz (PL); Karolina Sobczak, Zgierz (PL); Konrad Rudnicki, Lodz (PL); Karolina Kwaczynski, Lodz (PL); Slawomira Skrzypek, Lodz (PL)

(21) Appl. No.: 18/844,539

(22) PCT Filed: Nov. 9, 2022
(86) PCT No.: PCT/IB2022/060776

§ 371 Date: Sep. 6, 2024

(30) Foreign Application Priority Data
Mar. 7, 2022 (PL) 440575

(51) Int. Cl. G01N 27/416; G01N 33/94

(52) U.S. Cl. CPC G01N 27/416; G01N 33/946; G01N 33/9486

(57) ABSTRACT

The invention relates to an electrochemical method for determining heroin at liquid/liquid interfaces after dissolving its solid sample in aqueous solution. The method is characterised in that an electrochemical vessel made of a material resistant to organic solvents, equipped with two Ag/AgCl reference electrodes (7, 8) placed in Luggin capillaries (4, 5) and two auxiliary electrodes (6, 9) connected to the potentiostat (1), is filled with an organic phase (3) being a solution of a hydrophobic salt, up to half the distance between the Luggin capillary of the aqueous phase (4), and the Luggin capillary of the organic phase (5), and then the aqueous phase (2) being the heroin solution resulting from dissolving a sample containing heroin in a background electrolyte solution of the aqueous phase is poured. The liquid-liquid interface is formed between two Luggin capillaries (4, 5). In the next stage, measurements of the ionic current flow in the four-electrode system, which is simultaneously used to polarise the liquid-liquid interface created in this way, are carried out, using the potentiostat (1).

The present invention relates to a method for determining heroin that may be applied in forensic laboratories, in presumed analysis of street samples by law enforcement officers during the analysis of suspected street powders, or for parental control.

As observed by Rubin Gulaboski et al. (Rubin Gulaboski, M. Natalia D. S. Cordeiro, Nuno Milhazes, Jorge Garrido, Fernanda Borges, Miguel Jorge, Carlos M. Pereira, Ivan Bogeski, Aluska Helguera Morales, Blaze Naumoski, A. Fernando Silva, Anal. Biochem. 361 (2007) 236-243) heroin demonstrates activity at polar liquid interfaces. The authors of the paper determined for the first time the partition coefficients of ionised forms of several opioids (including heroin), amphetamine-like medicines and their metabolites by performing tests at the liquid/liquid interface.

Electrochemistry at liquid/liquid interfaces stands out from other electrochemical techniques because of its mechanism of analyte determination. There are conventional electrochemical sensors based on solid electrodes (e.g. carbon electrodes) that have been used for the determination of heroin. For these examples, the principle of operation is to record the currents associated with the electrochemical oxidation of heroin (oxidation of a tertiary amine that is part of the structure of the heroin molecule). Analysis of recorded signals allows for qualitative and quantitative determination of this substance (Jose R. Barreira Rodriguez, Victor Cabal Diaz, Agustin Costa Garcia and Paulino TuAon Blancs, Analyst, 115, 1990, 209-212; Anca Florea, Jonas Schram, Mats de Jong, Joy Eliaerts, Filip Van Durme, Balwinder Kaur, Nele Samyn, and Karolien De Wael, Anal. Chem., 91, 2019, 7920-7928).

Patent description PL/EP 2623987 discloses compositions and methods for detecting the presence and/or amount of one or more analytes, including analytes such as drugs of abuse. The compositions contain two or more analytes bound to a solid phase, e.g., a particle or a multiwell plate. The compositions and methods also allow for simultaneous, tandem, or serial determination of the presence and/or amount of two or more analytes of interest in a sample.

Patent description PL/EP 2283367 discloses methods that enable rapid release of one or more analytes from the head or body hair or other keratinised structures of an individual (who may have previously ingested one or more analytes). The methods may include contacting the keratinised structure with a reducing agent, but not a proteolytic agent. The methods may further include identifying and quantifying one or more analytes by known analytical techniques, such as immunoassays. The described methods do not damage the analyte and do not adversely affect the subsequently used analyte detection probe (e.g. an antibody).

American patent description U.S. Pat. No. 10,001,443 discloses a method for identifying the presence of heroin in an impure heroin composition that comprises heroin and at least one fluorescent impurity that interferes with the Raman signal from heroin. The method may include contacting the mixture with a solvent such as an alcohol, and then contacting the resulting alcohol composition with a SERS surface. The surface may then be exposed to laser light from a hand-held Raman spectrometer to detect a Raman signal from the heroin.

American patent U.S. Pat. No. 8,404,488 discloses an invention for the detection of heroin and morphine in illicit drug samples, as well as for their differentiation. A liquid sample being the dissolved street sample is prepared and divided into two equal portions, one being reference sample and other being main sample. Both samples are treated with hydrochloric acid and sodium hydroxide, but these substances are added to the samples in a different order. The Spectral Fluorescence Signatures (SFS) measurement of the reference sample is then performed. In addition, the presence of a specific spectral pattern of morphine in the measured SFS of the reference sample is detected and the value of the SFS intensity at a specific spectral point (reference value) is determined. After 15 minutes of acidification of the main sample, an analogous measurement and detection of morphine in the main sample is performed.

Based on the analysis of literature and patent databases, it can be concluded that so far no invention describing the use of polarised liquid-liquid interfaces for the detection and determination of heroin has been developed.

A method for electrochemical determining heroin according to the invention is characterised in that an electrochemical vessel made of a material resistant to organic solvents, preferably glass, equipped with two Ag/AgCl reference electrodes placed in Luggin capillaries and two auxiliary electrodes, preferably platinum ones, wherein the Ag/AgCl reference electrodes and auxiliary electrodes are connected to a potentiostat, is filled with an organic phase, which is a hydrophobic salt solution, preferably bis(triphenylphosphoranylidene)-ammonium tetrakis(4-chlorophenyl) borate, dissolved in a water-immiscible solvent characterised by dielectric properties allowing for at least partial dissociation of the hydrophobic salt into ions, preferably 1,2-dichloroethane. The organic phase is poured halfway between the Luggin capillary of the aqueous phase and the Lugin capillary of the organic phase. Then, the aqueous phase, which is a heroin solution resulting from the dissolution of the sample, which includes heroin in a background electrolyte solution of the aqueous phase, preferably NaCl, KCl or LiCl, with a concentration of several to several tens of millimoles per litre at pH 5.5 is added. A liquid-liquid interface is formed between two Luggin capillaries. In the next stage, measurements of the ion current flow in a four-electrode system are carried out using a potentiostat, and this flow is also used to polarise the liquid-liquid interface created in this way.

In another embodiment, the aqueous phase contains paracetamol with a concentration several thousand times greater than that of heroin.

In yet another embodiment, the aqueous phase contains caffeine with a concentration several thousand times higher than that of heroin.

In yet another version, the aqueous phase includes paracetamol and caffeine with a concentration several thousand times higher than that of heroin.

The pH of the aqueous phase ranges from pH 5.5 to pH 8.0 when paracetamol is present in the aqueous phase.

The pH of the aqueous phase ranges from pH 5.5 to pH 8.0 when caffeine is present in the aqueous phase.

The pH of the aqueous phase ranges from pH 5.5 to pH 8.0 when paracetamol and caffeine is present in the aqueous phase.

The advantage of the method according to the invention is that heroin can be determined in samples of street drugs containing paracetamol and/or caffeine after prior dissolution of the sample in the aqueous phase. The concentration of paracetamol and/or caffeine exceeding the concentration of heroin several thousand times does not affect the obtained electroanalytical values for the determination of heroin. The analysis is simple, takes several dozen seconds, allows for the detection of heroin at a concentration as low as of one micromole, the analysis can be integrated with mobile devices, and the analysis is selective-heroin can be determined in the presence of the most common impurities-caffeine and paracetamol.

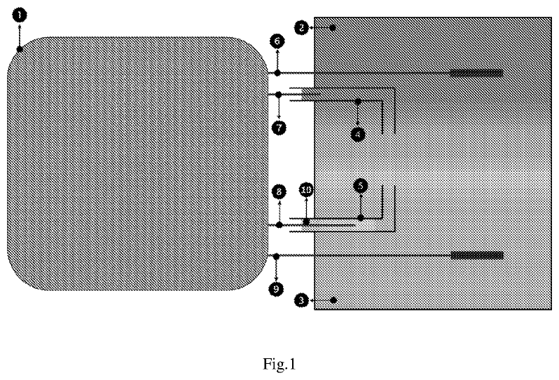

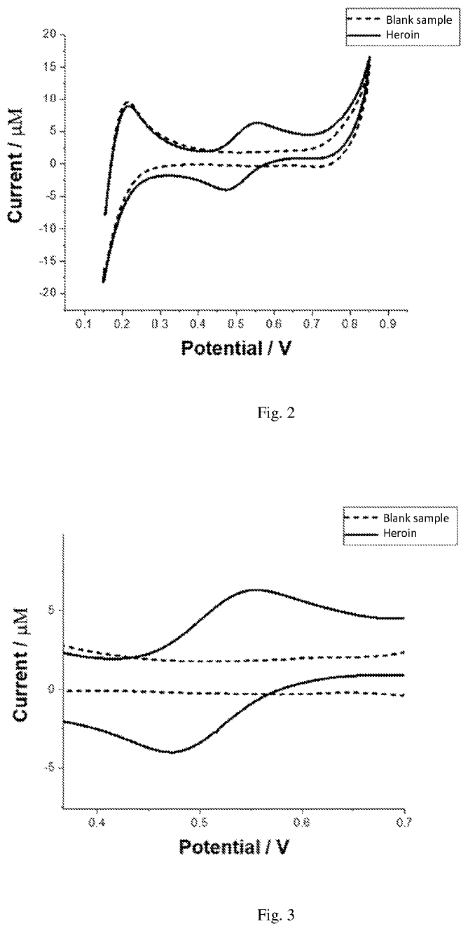

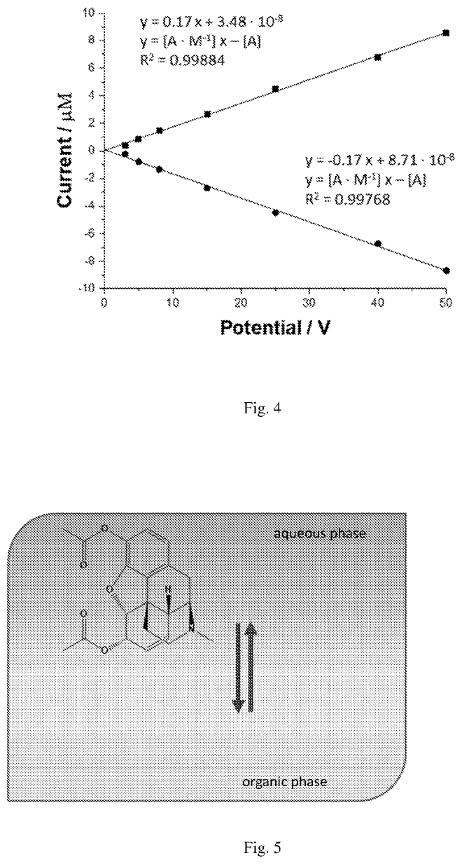

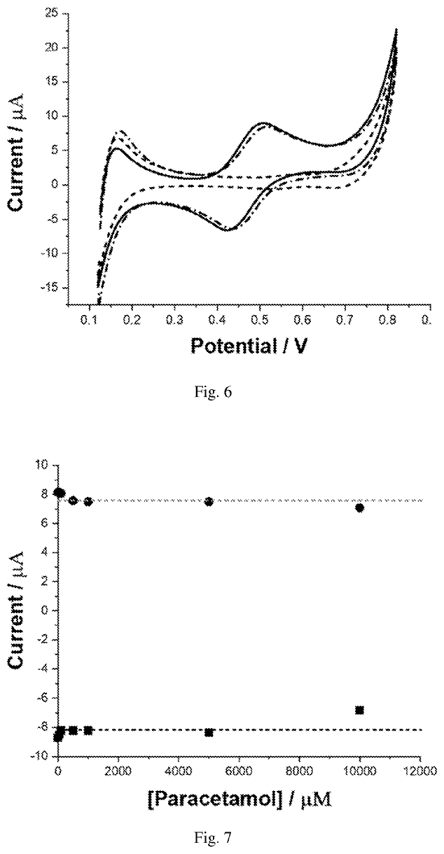

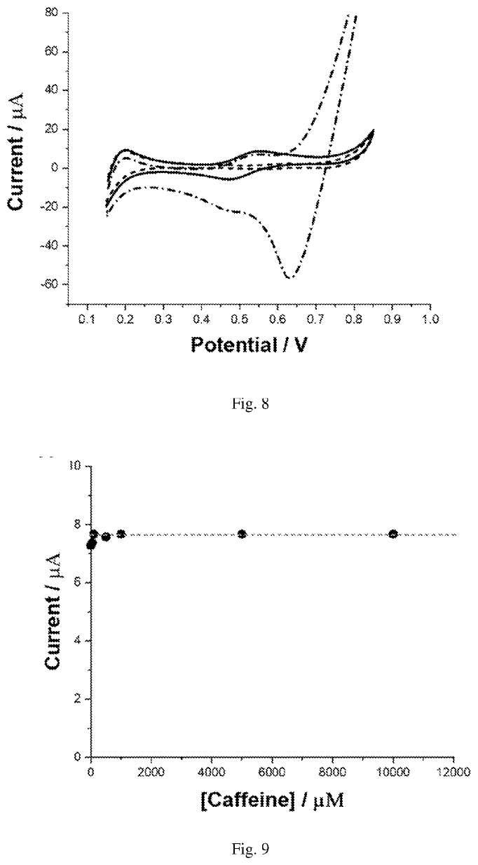

The subject of the invention has been presented in the examples and in the drawing, in which FIG. 1 presents a diagram of the measuring system used in the determination of heroin, FIG. 2 presents a cyclic voltammogram recorded for a blank sample containing no heroin in 10 mM HCl—a curve marked with a dashed line—and for 25 μM heroin dissolved in the aqueous phase—a solid line, FIG. 3 presents a framed signal from a voltammogram, FIG. 4 is a calibration curve plotted for heroin based on currents recorded at polarised liquid-liquid interfaces in the range of 1 to 50 μM, FIG. 5 presents a diagram of the interfacial transfer of heroin from the aqueous phase to the organic phase recorded as a Faradaic current, FIG. 6 presents cyclic voltammograms recorded for a blank sample in 10 mM NaCl with pH=5.5, dashed curve, for 50 μM concentration of heroin dissolved in the aqueous phase being 10 mM NaCl solution with pH=5.5, solid line, and for 50 μM concentration of heroin dissolved in the aqueous phase in 10 mM NaCl solution with pH=5.5 in the presence of 10 mM paracetamol, dashed-dotted curve, FIG. 7 is a summary showing the values of the positive and negative currents originating from the ionic interfacial transfer of heroin recorded in the presence of increasing concentrations of paracetamol in the case when the aqueous phase was 10 mM NaCl with pH=5.5, FIG. 8 shows cyclic voltammograms recorded for a blank sample of the aqueous phase being 10 mM NaCl with pH=5.5, dashed curve, for 50 μM concentration of heroin dissolved in the aqueous phase being 10 mM NaCl solution with pH=5.5, solid line, and for 50 μM concentration of heroin dissolved in the aqueous phase being 10 mM NaCl solution with pH=5.5 in the presence of 10 mM caffeine, dashed-dotted curve and FIG. 9 presents a summary of the values of the positive currents originating from the ionic interfacial transfer of heroin recorded in the presence of increasing concentrations of caffeine, the concentration of which is marked on the x-axis.

### EXAMPLE 1

A properly constructed electrochemical vessel made of glass or other material resistant to organic solvents consists of a part filled with the organic phase 3 being a solution of bis(triphenylphosphorylidene)ammonium tetrakis-4-chlorophenylborate dissolved in 1,2-dichloroethane and the aqueous phase 2 being a solution of hydrogen chloride at a concentration of 10 mM. The vessel is equipped with a set of electrodes. Two reference electrodes-Ag/AgCl 7 and 8 and two auxiliary platinum electrodes 6 and 9, each immersed in one of the phases, were used. An aqueous solution containing heroin was poured into the vessel to which the organic phase had previously been added. A liquid-liquid interface is formed between two Luggin capillaries 4 and 5. In the next stage, potentiostat 1 was used, allowing for measurements in a four-electrode system, in order to polarise the liquid-liquid phase interface created in this way. Cyclic voltammetry is an example of a method for recording current-voltage signals on the basis of which a calibration curve for the tested analyte is plotted. The dashed line plot was recorded in a heroin-free model solution, the solid line plot was recorded at a heroin concentration of 25 μM. The analysis of current-potential relationships carried out using a dedicated software recording current-potential relationships allows for qualitative and quantitative determination of heroin. The difference in the Galvani potential of the transfer of the heroin molecule from the aqueous phase to the organic phase is a constant value for the applied physicochemical parameters, which is the basis for qualitative analysis. The faradaic currents of the positive peak originating from the transfer of the ionised heroin molecule from the aqueous phase to the organic phase or of the negative peak originating from the transfer of the ionised heroin molecule from the organic phase to the aqueous phase are directly proportional to the concentration of heroin in the aqueous phase. Heroin concentration is determined based on calibration methods.

### EXAMPLE 2

The procedure was the same as in Example 1 except that a solution of bis(triphenylphosphorylidene)ammonium tetrakis-3,5-bis(trifluoromethyl)phenylborate dissolved in nitrobenzene was used as the organic phase.

### EXAMPLE 3

The procedure was the same as in Example 1 except that a solution of tetrakis(4-chlorophenyl) tetradodecylammonium borate dissolved in 2-nitrophenyl n-octyl ether was used as the organic phase.

1. A method for determining heroin, comprising: providing an electrochemical vessel made of a material resistant to organic solvents, equipped with two reference electrodes placed in Luggin capillaries and two-auxiliary electrodes, wherein the reference electrodes and auxiliary electrodes are connected to a potentiostat, is filled with an organic phase being a solution of a hydrophobic salt, preferably bis(triphenylphosphorylidene)ammonium tetrakis-4-chlorophenylborate, dissolved in a water-immiscible solvent with dielectric properties allowing for at least partial dissociation of the hydrophobic salt into ions, preferably 1,2-dichloroethane, up to half of a distance between the Luggin capillary of aqueous phase, and the Luggin capillary of the organic phase, and then aqueous phase being the heroin solution resulting from dissolving a sample containing heroin in a background electrolyte solution of the aqueous phase, preferably NaCl, KCl or LiCl, with a concentration of several to several tens of millimoles per litre at pH 5.5 is poured, wherein a liquid-liquid interface is formed between two Luggin capillaries, and then, in a next stage, measurements of ionic current flow in four-electrode system, which is simultaneously used to polarize the liquid-liquid interface created in this way, are carried out, using the potentiostat.

2. The method according to claim 1, wherein the aqueous phase contains paracetamol with a concentration several thousand times higher than that of heroin.

3. The method according to claim 1, wherein the aqueous phase contains caffeine with a concentration several thousand times higher than that of heroin.

4. The method according to claim 1, wherein the aqueous phase contains paracetamol and caffeine with a concentration several thousand times higher than that of heroin.

5. The method according to claim 2, wherein the pH of the aqueous phase is from pH 5.5 to pH 8.0 in case paracetamol is present in the aqueous phase.

6. The method according to claim 3, wherein the pH of the aqueous phase is from pH 5.5 to pH 8.0 in case caffeine is present in the aqueous phase.

7. The method according to claim 4, wherein the pH of the aqueous phase is from pH 5.5 to pH 8.0 in case paracetamol and caffeine are present in the aqueous phase.

8. The method according to claim 1, wherein the electrochemical vessel is preferably made out of glass.

9. The method according to claim 1, wherein the auxiliary electrodes are preferably made out of platinum.

10. The method according to claim 1, wherein the reference electrodes are preferably made out of Ag covered with AgCl layer.
